(19) United States
(12) Patent Application Publication
(10) Pub. No.: US 2025/0266173 A1
(43) Pub. Date: Aug. 21, 2025

(54) SYMPTOM UNDERSTANDING ASSIST APPARATUS, SYMPTOM UNDERSTANDING ASSIST METHOD, AND RECORDING MEDIUM

(71) Applicant: NEC Corporation, Tokyo (JP)

(72) Inventors: Hirokazu Sakamoto, Tokyo (JP); Tomoharu Kiyuna, Tokyo (JP); Tatsu Kimura, Tokyo (JP)

(73) Assignee: NEC Corporation, Tokyo (JP)

(21) Appl. No.: 19/047,855

(22) Filed: Feb. 7, 2025

(30) Foreign Application Priority Data
Feb. 16, 2024 (JP) 2024-022397

(51) Int. Cl. G16H 70/20; G16H 10/60; G16H 30/00; G16H 50/20; G16H 80/00

(52) U.S. Cl. CPC G16H 70/20; G16H 10/60; G16H 30/00; G16H 50/20; G16H 80/00

(57) ABSTRACT

This symptom understanding assist apparatus includes: an explanation acquiring section for acquiring explanatory text of an explanation made by a patient about a symptom in the patient; a summary generating section for generating summary text of the explanatory text with use of a large language model with the explanatory text used as an input; a medical information acquiring section for acquiring medical information regarding the patient; a symptom inferring section for inferring, based on the summary text and the medical information, a symptom candidate for the symptom in the patient; and an outputting section for outputting the summary text and the symptom candidate inferred.

### CROSS REFERENCE TO RELATED APPLICATIONS

This application is based upon and claims the benefit of priority from Japanese Patent Application No. 2024-022397 filed on Feb. 16, 2024, the disclosure of which is incorporated herein in its entirety by reference.

### TECHNICAL FIELD

The present disclosure relates to a symptom understanding assist apparatus, a symptom understanding assist method, and a recording medium.

### BACKGROUND ART

Diagnosis assist techniques are known. Examples of diagnosis assist techniques include the technique disclosed in Patent Literature 1. Patent Literature 1 discloses an information processing apparatus for: acquiring patient's medical information including at least one of pieces of information which are patient information regarding a patient having undergone a medical interview and medical interview information regarding answers to questions in the medical interview; extracting, based on the medical information, at least one piece of information from information group which at least includes treatment information regarding a treatment associated with each of the pieces of information and drug information regarding drugs; and outputting medical-related information regarding the extracted information and the medical information, to another information processing apparatus.

### CITATION LIST

### Patent Literature

[Patent Literature 1]

Japanese Patent Application Publication Tokukai No. 2022-142234

### SUMMARY OF INVENTION

### Technical Problem

In a medical examination, there are a case where patient's verbal explanation is verbose and off target and a case where a patient is a child or the like and cannot accurately explain their subjective symptom. In such cases, where it is difficult to accurately understand the content of the verbal explanation of a patient, there is a problem of being impossible for a medical service worker, such as a doctor who diagnoses a patient, to properly diagnose the patient through the technique disclosed in Patent Literature 1.

The present disclosure has been made in view of the above problem, and an example object thereof is to provide a technique which makes it possible to more properly assist in understanding a symptom of a patient.

### Solution to Problem

A symptom understanding assist apparatus in accordance with an example aspect of the present disclosure includes at least one processor, and the at least one processor carries out: an explanation acquiring process of acquiring explanatory text of an explanation made by a patient about a symptom in the patient; a summary generating process of generating summary text of the explanatory text with use of a language model with the explanatory text used as an input; a medical information acquiring process of acquiring medical information regarding the patient; a symptom inferring process of inferring, based on the summary text and the medical information, a symptom candidate for the symptom in the patient; and an outputting process of outputting the summary text and the symptom candidate inferred.

A symptom understanding assist method in accordance with an example aspect of the present disclosure includes: an explanation acquiring process of at least one processor acquiring explanatory text of an explanation made by a patient about a symptom in the patient; a summary generating process of the at least one processor generating summary text of the explanatory text with use of a language model with the explanatory text used as an input; a medical information acquiring process of the at least one processor acquiring medical information regarding the patient; a symptom inferring process of the at least one processor inferring, based on the summary text and the medical information, a symptom candidate for the symptom in the patient; and an outputting process of the at least one processor outputting the summary text and the symptom candidate inferred.

A recording medium in accordance with an example aspect of the present disclosure is a recording medium having recorded thereon a symptom understanding assist program for causing a computer to function as a symptom understanding assist apparatus, the symptom understanding assist program causing the computer to carry out: an explanation acquiring process of acquiring explanatory text of an explanation made by a patient about a symptom in the patient; a summary generating process of generating summary text of the explanatory text with use of a language model with the explanatory text used as an input; a medical information acquiring process of acquiring medical information regarding the patient; a symptom inferring process of inferring, based on the summary text and the medical information, a symptom candidate for the symptom in the patient; and an outputting process of outputting the summary text and the symptom candidate inferred.

### Advantageous Effects of Invention

An example aspect of the present disclosure provides an example advantage of making it possible to provide a technique to more properly assist in understanding a symptom of a patient.

### BRIEF DESCRIPTION OF DRAWINGS

FIG. 1 is a block diagram illustrating a configuration of a symptom understanding assist apparatus in accordance with the present disclosure.

FIG. 2 is a flowchart illustrating a flow of a symptom understanding assist method in accordance with the present disclosure.

FIG. 3 is a block diagram illustrating a configuration of an information processing apparatus in accordance with the present disclosure.

FIG. 4 is a diagram illustrating a functional configuration of a control section in accordance with the present disclosure.

FIG. 5 is a diagram illustrating a specific example of information outputted by an output control section in accordance with the present disclosure.

FIG. 6 is a flowchart illustrating an example flow of the symptom understanding assist method in accordance with the present disclosure.

FIG. 7 is a block diagram illustrating a configuration of a computer which functions as the symptom understanding assist apparatus or the information processing apparatus in accordance with the present disclosure.

### EXAMPLE EMBODIMENTS

The following description will discuss example embodiments of the present invention. However, the present invention is not limited to the example embodiments described below, but can be altered by a skilled person in the art within the scope of the claims. For example, any embodiment derived by appropriately combining techniques (some or all of products or methods) adopted in differing example embodiments described below can be within the scope of the present invention. Further, any embodiment derived by appropriately omitting one or more of the techniques adopted in differing example embodiments described below can be within the scope of the present invention. Furthermore, the advantage mentioned in each of the example embodiments described below is an example advantage expected in that example embodiment, and does not define the extension of the present invention. That is, any embodiment which does not provide the example advantages mentioned in the example embodiments described below can also be within the scope of the present invention.

### First Example Embodiment

The following description will discuss a first example embodiment, which is an example embodiment of the present invention, in detail with reference to the drawings. The present example embodiment is basic to each of the example embodiments which will be described later. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### (Configuration of Symptom Understanding Assist Apparatus)

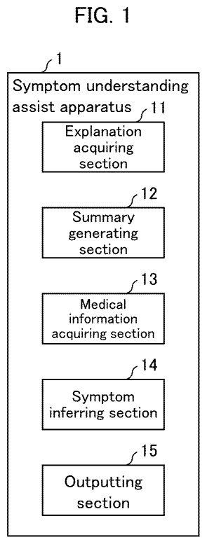

The configuration of a symptom understanding assist apparatus 1 is described here with reference to FIG. 1. FIG. 1 is a block diagram illustrating the configuration of the symptom understanding assist apparatus 1. The symptom understanding assist apparatus 1 includes an explanation acquiring section 11, a summary generating section 12, a medical information acquiring section 13, a symptom inferring section 14, and an outputting section 15, as illustrated in FIG. 1. The explanation acquiring section 11 acquires explanatory text of an explanation made by a patient about a symptom in the patient. The summary generating section 12 generates summary text of the explanatory text with use of a language model with the explanatory text used as an input. The medical information acquiring section 13 acquires medical information regarding the patient. The symptom inferring section 14 infers, based on the summary text and the medical information, a symptom candidate for the symptom in the patient. The outputting section 15 outputs the summary text and the symptom candidate inferred.

### (Example Advantage of Symptom Understanding Assist Apparatus)

As above, the symptom understanding assist apparatus 1 includes: an explanation acquiring section 11 for acquiring explanatory text of an explanation made by a patient about a symptom in the patient; a summary generating section 12 for generating summary text of the explanatory text with use of a language model with the explanatory text used as an input; a medical information acquiring section 13 for acquiring medical information regarding the patient; a symptom inferring section 14 for inferring, based on the summary text and the medical information, a symptom candidate for the symptom in the patient; and an outputting section 15 for outputting the summary text and the symptom candidate inferred. Thus, the symptom understanding assist apparatus 1 provides an example advantage of making it possible to more properly assist in understanding a symptom in a patient.

### (Flow of Symptom Understanding Assist Method)

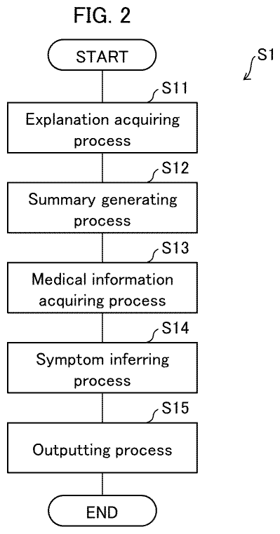

The flow of a symptom understanding assist method S1 is described here with reference to FIG. 2. FIG. 2 is a flowchart illustrating the flow of the symptom understanding assist method S1. The symptom understanding assist method S1 includes an explanation acquiring process S11, a summary generating process S12, a medical information acquiring process S13, a symptom inferring process S14, and an outputting process S15, as illustrated in FIG. 2. In the explanation acquiring process S11, at least one processor acquires explanatory text of an explanation made by a patient about a symptom in the patient. In the summary generating process S12, the at least one processor generates summary text of the explanatory text with use of a language model with the explanatory text used as an input. In the medical information acquiring process S13, the at least one processor acquires medical information regarding the patient. In the symptom inferring process S14, the at least one processor infers, based on the summary text and the medical information, a symptom candidate for the symptom in the patient. In the outputting process S15, the at least one processor outputs the summary text and the symptom candidate inferred.

### (Example Advantage of Symptom Understanding Assist Method)

As above, the symptom understanding assist method S1 includes: an explanation acquiring process S11 of at least one processor acquiring explanatory text of an explanation made by a patient about a symptom in the patient; a summary generating process S12 of the at least one processor generating summary text of the explanatory text with use of a language model with the explanatory text used as an input; a medical information acquiring process S13 of the at least one processor acquiring medical information regarding the patient; a symptom inferring process S14 of the at least one processor inferring, based on the summary text and the medical information, a symptom candidate for the symptom in the patient; and an outputting process S15 of the at least one processor outputs the summary text and the symptom candidate inferred. Thus, the symptom understanding assist method S1 provides an example advantage of making it possible to more properly assist in understanding a symptom in a patient.

### Second Example Embodiment

The following description will discuss a second example embodiment, which is an example embodiment of the present invention, in detail with reference to the drawings. A component having the same function as a component described in the above example embodiment is assigned the same reference sign, and the description thereof is omitted where appropriate. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### (Configuration of Information Processing Apparatus)

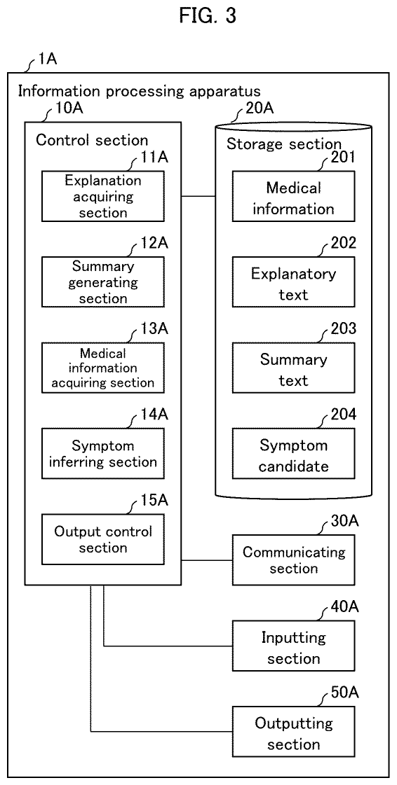

An information processing apparatus 1A outputs information for assisting in understanding a symptom in a patient. The information processing apparatus 1A is an example of the symptom understanding assist apparatus in accordance with the present disclosure. The configuration of the information processing apparatus 1A is described here with reference to FIG. 3. FIG. 3 is a block diagram illustrating the configuration of the information processing apparatus 1A. The information processing apparatus 1A includes a control section 10A, a storage section 20A, a communicating section 30A, an inputting section 40A, and an outputting section 50A.

### (Communicating Section)

The communicating section 30A communicates with an apparatus external to the information processing apparatus 1A over a communication line. A specific configuration of the communication line does not limit the present example embodiment, but examples of the communication line include a wireless local area network (LAN), a wired LAN, a wide area network (WAN), a public network, a mobile data communication network, and a combination thereof. The communicating section 30A transmits, to another apparatus, data supplied from the control section 10A, and supplies the control section 10A with data received from another apparatus.

### (Inputting Section)

The inputting section 40A is a component for accepting an input to the information processing apparatus 1A, and includes inputting equipment such as, for example, a keyboard, a mouse, a touch panel, a camera, or a microphone. Further, the inputting section 40A may be a component for accepting data from inputting equipment via an interface such as, for example, a universal serial bus (USB).

### (Outputting Section)

The outputting section 50A is a component for producing an output from the information processing apparatus 1A, and includes outputting equipment such as, for example, a display, a printer, a touch panel, or a speaker. The outputting section 50A may be a component which, for example, includes an interface such as a USB and outputs data to outputting equipment via the interface.

### (Storage Section)

In the storage section 20A, various kinds of information to be referred to by the control section 10A are stored. Examples of such information include medical information 201, explanatory text 202, summary text 203, and a symptom candidate 204.

The medical information 201 is information related to medical care for a patient. Examples of the medical information 201 include medical record information. As an example, the medical information 201 includes at least one selected from the group consisting of personal information regarding the patient, information on findings shown by a medical examination performed on the patient, and medical history information regarding the patient. The personal information regarding the patient is personal information regarding the patient, and includes, for example, information indicating the age, the gender, the amount of smoking, etc. of the patient. The information on findings shown by a medical examination performed on the patient is information indicating findings made by a medical service worker, such as a doctor, and includes, for example, information obtained by the medical service worker through a medical interview or an inspection. The information obtained through a medical interview or an inspection may be, for example, text “pressure on the abdomen causes a pain”. The medical history information regarding the patient is information regarding the history of disease of the patient, and includes, for example, information indicating the past medical history of the patient, the past medical history of the family, the amount of smoking of the patient, etc. In addition, the medical information 201 may include information indicating the measurement results of vital signs of the patient. The information indicating the measurement results of vital signs may be, for example, information indicating “37.8° C./heart rate 90/breathing rate 30”.

The medical information 201 may include a diagnosis target image of the patient. As an example, the diagnosis target image includes at least one of images which are an X-ray image, an endoscope image, a pathological image, an MRI image, and a CT image. In the storage section 20A, respective pieces of medical information 201 regarding a plurality of patients are stored.

The explanatory text 202 is text representing the content of an explanation made by the patient about a symptom in the patient. For example, the explanatory text 202 may be text representing the content of a verbal explanation made by the patient about a symptom in the patient, or may be text representing the content of what the patient has written on a medical interview sheet or the like regarding a symptom in the patient. In a case where a patient verbally explains a symptom in the patient, the process of converting a speech made by the patient into text is carried out by, for example, an explanation acquiring section 11A, which will be described later. The explanatory text 202 is, for example, text “I feel sluggish and have a pain in the central area of the belly. I have been feeling sick and feverish . . . ”.

The summary text 203 is text generated by a summary generating section 12A, which will be described later. The summary text 203 represents the content of a summary of the explanatory text 202. The summary text 203 is, for example, text “sluggishness. pain in the solar plexus area. Sick feeling and slight fever”.

The symptom candidate 204 is information indicating a symptom in the patient, the symptom being inferred by a symptom inferring section 14A, which will be described later. For example, the symptom candidate 204 is information such as “appendicitis/acute gastritis”.

### (Control Section)

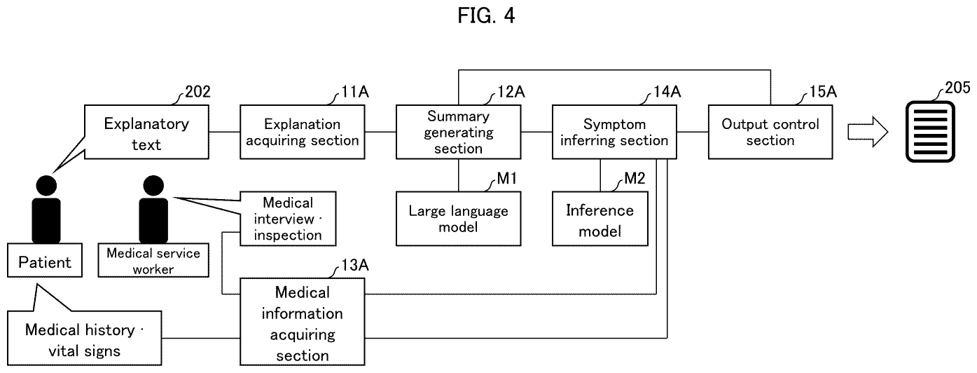

FIG. 4 is a diagram illustrating a functional configuration of the control section 10A. The control section 10A includes an explanation acquiring section 11A, a summary generating section 12A, a medical information acquiring section 13A, a symptom inferring section 14A, and an output control section 15A.

### (Explanation Acquiring Section)

The explanation acquiring section 11A acquires the explanatory text 202 of an explanation made by the patient about a symptom of the patient, and provides the summary generating section 12A with the explanatory text 202 acquired. As an example, the explanation acquiring section 11A may acquire the explanatory text 202 by retrieving the explanatory text 202 from storage location (which may be storage in the information processing apparatus 1A, or may be storage external to the information processing apparatus 1A) designated by a user of the information processing apparatus 1A. The explanation acquiring section 11A may acquire the explanatory text 202 by receiving the explanatory text 202 from another apparatus via the communicating section 30A. The explanation acquiring section 11A may acquire the explanatory text 202 inputted to the inputting section 40A.

The explanation acquiring section 11A may acquire speech data which represents a speech picked up in a medical examination performed on the patient and convert the acquired speech data into text data.

### (Summary Generating Section)

The summary generating section 12A generates summary text of the explanatory text 202 with use of a large language model M1 with the explanatory text 202 used as an input. The large language model M1 is an example of the language model in accordance with the present disclosure. The large language model M1 may be stored in the storage section 20A of the information processing apparatus 1A, or may be stored in an apparatus other than the information processing apparatus 1A. The large language model M1 being stored in the storage section 20A means that parameters defining the large language model M1 are stored in the storage section 20A.

The large language model M1 is a language model which is formed by an artificial neural network having a great number of parameters and which is generated by machine learning. Examples of the large language model M1 includes, but is not limited to, generative AI such as Chat Generative Pre-Trained Transformer (ChatGPT) or Generative Pre-trained Transformer 4 (GPT-4), and such generative AI having been fine-tuned with data related to medical care.

Input information to be inputted by the summary generating section 12A to the large language model M1 includes the explanatory text 202. Further, the input information may include information (e.g. text) indicating summary generation instructions. Output information to be outputted from the large language model M1 includes the summary text 203, which is a summary of the explanatory text 202.

As an example, in a case where the large language model M1 is stored in an apparatus other than the information processing apparatus 1A, the summary generating section 12A transmits the explanatory text 202 via the communicating section 30A to the apparatus in which the large language model M1 is stored, to input the explanatory text 202 to the large language model M1. In this case, the summary generating section 12A receives information outputted by the large language model M1 from that apparatus via the communicating section 30A.

### (Medical Information Acquiring Section)

The medical information acquiring section 13A acquires the medical information 201 regarding the patient. In the example of FIG. 4, the medical information acquiring section 13A acquires the medical information 201 which includes: information obtained through a medical interview and an inspection; information indicating a medical history; and information indicating vital signs. As an example, the medical information acquiring section 13A may acquire the medical information 201 by retrieving the medical information 201 from a storage location (which may be storage in the information processing apparatus 1A, or may be storage external to the information processing apparatus 1A) designated by a user of the information processing apparatus 1A. The medical information acquiring section 13A may acquire the medical information 201 by receiving the medical information 201 from another apparatus via the communicating section 30A. The medical information acquiring section 13A may acquire the medical information 201 inputted to the inputting section 40A.

### (Symptom Inferring Section)

The symptom inferring section 14A infers, based on the summary text 203 and the medical information 201, a symptom candidate for a symptom in the patient. The symptom candidate is used in, for example, decision making on diagnosis of the patient.

As an example, the symptom inferring section 14A infers a symptom candidate, based on an output obtained by inputting the summary text 203 and the medical information 201 to an inference model M2. The inference model M2 is a trained model generated by machine learning. Examples of the inference model M2 include, but is not limited to, a trained model generated by supervised learning with use of a neural network approach or the like, generative AI such as ChatGPT or GPT-4, and such generative AI having been fine-tuned with data related to medical care. The inference model M2 and the large language model M1 may be one and the same.

The inference model M2 may be stored in the storage section 20A of the information processing apparatus 1A, or may be stored in an apparatus other than the information processing apparatus 1A. The inference model M2 being stored in the storage section 20A means that parameters defining the inference model M2 are stored in the storage section 20A.

Input information to be inputted by the symptom inferring section 14A to the inference model M2 includes the summary text 203 and the medical information 201. Output information to be outputted from the inference model M2 is information indicating an inference result (also referred to as “symptom candidate”) of inference of a symptom in the patient. For example, the information indicating an inference result is information indicating “appendicitis/acute gastritis”.

As an example, in a case where the inference model M2 is stored in an apparatus other than the information processing apparatus 1A, the symptom inferring section 14A transmits the summary text 203 and the medical information 201 via the communicating section 30A to the apparatus in which the inference model M2 is stored, to input the summary text 203 and the medical information 201 to the inference model M2. In this case, the symptom inferring section 14A receives information outputted by the inference model M2 from that apparatus via the communicating section 30A.

### (Output Control Section)

The output control section 15A outputs output information 205 which includes the summary text 203 and the symptom candidate 204. As an example, the output control section 15A may output the output information 205 by writing the output information 205 in a storage location (which may be storage in the information processing apparatus 1A, or may be storage external to the information processing apparatus 1A) designated by a user of the information processing apparatus 1A. Further, the output control section 15A may transmit the output information 205 via the communicating section 30A, or may output the output information 205 to outputting equipment such as a display.

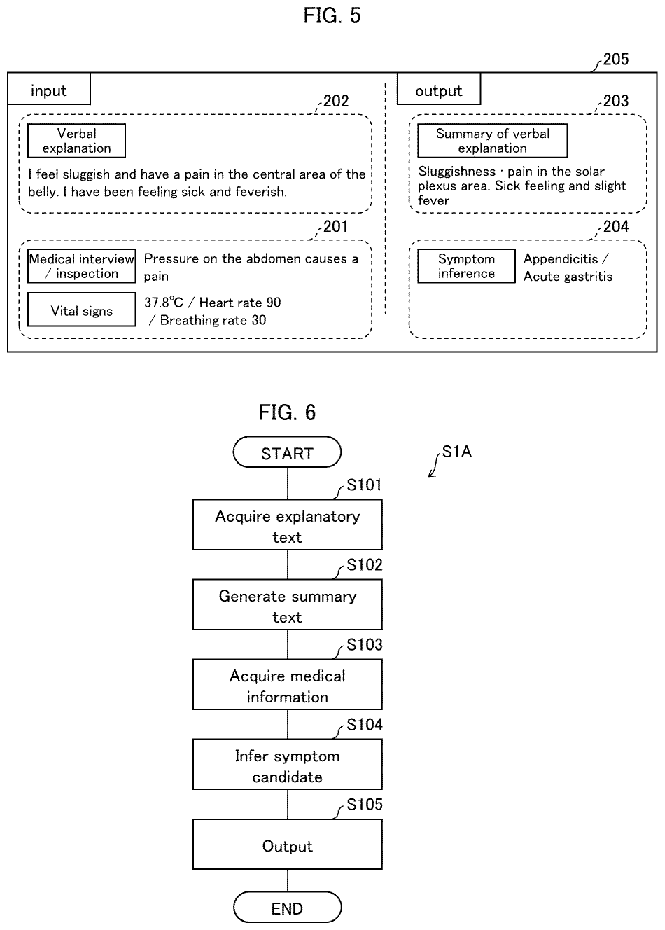

FIG. 5 is a diagram illustrating a specific example of the output information 205 outputted by the output control section 15A. As an example, the output control section 15A may display the output information 205 illustrated in FIG. 5 on displaying equipment. In this case, a screen displayed on the displaying equipment includes the summary text 203 and the symptom candidate 204. The screen further includes the explanatory text 202 and the medical information 201, which are inputs to the information processing apparatus 1A. As illustrated in FIG. 5, the output control section 15A may output the explanatory text 202 and the medical information 201 together with the summary text 203 and the symptom candidate 204.

In the example of FIG. 5, the output information 205 includes the explanatory text 202 of a verbal explanation “I feel sluggish and have a pain in the central area of the belly. I have been feeling sick and feverish . . . ”. The output information 205 includes the medical information 201 which includes: information “pressure on the abdomen causes a pain” obtained through a medical interview or an inspection; and vital sign information “37.8° C./heart rate 90/breathing rate 30”. The output information 205 includes the summary text 203 of the verbal explanation “sluggishness. pain in the solar plexus area. Sick feeling and slight fever”. The output information 205 includes the symptom candidate 204 “appendicitis/acute gastritis” inferred.

### (Flow of Symptom Understanding Assist Method)

FIG. 6 is a flowchart illustrating an example flow of a symptom understanding assist method S1A carried out by the information processing apparatus 1A. The steps included in the flowchart of FIG. 6 may be carried out in parallel with each other or in a different order.

In step S101, the explanation acquiring section 11A acquires the explanatory text 202. In step S102, the summary generating section 12A generates the summary text 203 of the explanatory text 202 with use of the large language model M1. In step S103, the medical information acquiring section 13A acquires the medical information 201. In step S104, the symptom inferring section 14A infers a symptom candidate for a symptom in a patient, by inputting the summary text 203 and the medical information 201 to the inference model M2. In step S105, the output control section 15A outputs the summary text 203 and the symptom candidate inferred in step S104.

### (Example Advantage of Information Processing Apparatus)

As above, the information processing apparatus 1A infers a symptom in a patient, with use of not only the medical information 201 regarding the patient but also the summary text 203, which is a summary of the explanatory text 202 of an explanation made by the patient about the symptom in the patient. With this configuration, even in a case where, for example, patient's verbal explanation is verbose and off target, it is possible to more properly infer, via the information processing apparatus 1A, a symptom in a patient. This makes it possible to more properly assist in understanding the symptom in the patient.

In the information processing apparatus 1A, the medical information 201 includes at least one selected from the group consisting of personal information regarding a patient, information on findings shown by a medical examination performed on the patient, and medical history information regarding the patient. Thus, with the information processing apparatus 1A, it is possible to more properly infer a symptom in a patient with use of at least one selected from the group consisting of personal information regarding the patient, information on findings shown by a medical examination performed on the patient, and medical history information regarding the patient.

In the information processing apparatus 1A, the explanation acquiring section 11A converts a speech picked up in a medical examination performed on a patient into text data. Thus, with the information processing apparatus 1A, it is possible to more properly infer a symptom in a patient, only by performing a medical examination on the patient. That is, the patient only need to undergo a medical examination and does not need to carry out any other complicated tasks.

In the information processing apparatus 1A, the symptom inferring section 14A infers a symptom candidate for a symptom in a patient, based on an output obtained by inputting the summary text 203 and the medical information 201 to the inference model M2 generated by machine learning. Thus, with the information processing apparatus 1A, it is possible to more properly infer a symptom in a patient, by using the inference model M2 generated by machine learning.

In the information processing apparatus 1A, the medical information 201 includes a diagnosis target image of a patient. By using a diagnosis target image of the patient, it is possible to more properly infer a symptom in the patient.

In the information processing apparatus 1A, the symptom candidate 204 is information to be used in decision making on diagnosis of a patient. This enables a medical service worker or the like who diagnoses a patient to more properly carry out decision making on diagnosis.

### Software Implementation Example

Some or all of the functions of the symptom understanding assist apparatus 1 and the information processing apparatus 1A (hereinafter, also referred to as “each apparatus above”) may be implemented by hardware such as an integrated circuit (IC chip), or may be implemented by software.

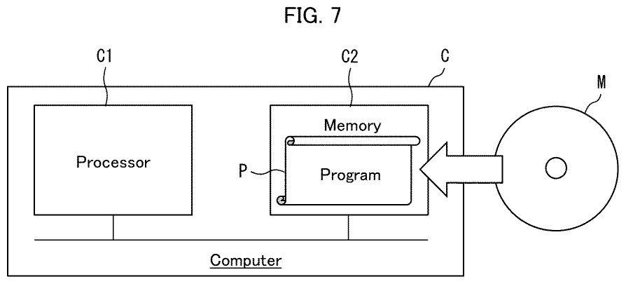

In the latter case, the each apparatus above is provided by, for example, a computer that executes instructions of a program that is software implementing the functions. An example (hereinafter, computer C) of such a computer is illustrated in FIG. 7. FIG. 7 is a block diagram illustrating a hardware configuration of the computer C which functions as each apparatus above.

The computer C includes at least one processor C1 and at least one memory C2. The memory C2 has recorded thereon a program P for causing the computer C to operate as each apparatus above. The processor C1 of the computer C retrieves the program P from the memory C2 and executes the program P, so that the functions of each apparatus above are implemented.

Examples of the processor C1 can include a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Examples of the memory C2 can include a flash memory, a hard disk drive (HDD), a solid state drive (SSD), and a combination thereof.

The computer C may further include a random access memory (RAM) into which the program P is loaded at the time of execution and in which various kinds of data are temporarily stored. The computer C may further include a communication interface via which data is transmitted to and received from another apparatus. The computer C may further include an input-output interface via which inputting-outputting equipment such as a keyboard, a mouse, a display, or a printer is connected.

The program P can be recorded on a non-transitory tangible recording medium M capable of being read by the computer C. Examples of such a recording medium M can include a tape, a disk, a card, a semiconductor memory, and a programmable logic circuit. The computer C can obtain the program P via such a recording medium M. The program P can be transmitted via a transmission medium. Examples of such a transmission medium can include a communication network and a broadcast wave. The computer C can obtain the program P also via such a transmission medium.

The above-described functions of each apparatus above may be implemented by a single processor provided in a single computer, may be implemented by the cooperation among a plurality of processors provided in a single computer, or may be implemented by the cooperation among a plurality of processors provided in a plurality of respective computers. Further, the program for causing each apparatus above to implement the above-described functions may be stored in a single memory provided in a single computer, may be stored in a distributed manner in a plurality of memories provided in a single computer, or may be stored in a distributed manner in a plurality of memories provided in a plurality of respective computers.

### Additional Remark A

The whole or part of the example embodiments disclosed above can be described as, but not limited to, the following supplementary notes.

### (Supplementary Note A1)

A symptom understanding assist apparatus, including: an explanation acquiring means for acquiring explanatory text of an explanation made by a patient about a symptom in the patient;
a summary generating means for generating summary text of the explanatory text with use of a large language model with the explanatory text used as an input;
a medical information acquiring means for acquiring medical information regarding the patient;
a symptom inferring means for inferring, based on the summary text and the medical information, a symptom candidate for the symptom in the patient; and
an outputting means for outputting the summary text and the symptom candidate inferred.

### (Supplementary Note A2)

The symptom understanding assist apparatus described in supplementary note A1, in which the medical information includes at least one selected from the group consisting of personal information regarding the patient, information on findings shown by a medical examination performed on the patient, and medical history information regarding the patient.

### (Supplementary Note A3)

The symptom understanding assist apparatus described in supplementary note A1 or A2, in which the explanation acquiring means is configured to convert, into text data, a speech picked up in the medical interview performed on the patient.

### (Supplementary Note A4)

The symptom understanding assist apparatus described in any one of supplementary notes A1 to A3, in which the symptom inferring means is configured to infer the symptom candidate, based on an output obtained by inputting the summary text and the medical information to a trained model generated by machine learning.

### (Supplementary Note A5)

The symptom understanding assist apparatus described in any one of supplementary notes A1 to A4, in which the medical information includes a diagnosis target image of the patient.

### (Supplementary Note A6)

The symptom understanding assist apparatus described in any one of supplementary notes A1 to A5, in which the symptom candidate is information to be used in decision making on diagnosis of the patient.

### Additional Remark B

The whole or part of the example embodiments disclosed above can be described as, but not limited to, the following supplementary notes.

### (Supplementary Note B1)

A symptom understanding assist method, including: at least one processor acquiring explanatory text of an explanation made by a patient about a symptom in the patient;
the at least one processor generating summary text of the explanatory text with use of a large language model with the explanatory text used as an input;
the at least one processor acquiring medical information regarding the patient;
the at least one processor inferring, based on the summary text and the medical information, a symptom candidate for the symptom in the patient; and
the at least one processor outputting the summary text and the symptom candidate inferred.

### (Supplementary Note B2)

The symptom understanding assist method described in supplementary note B1, in which the medical information includes at least one selected from the group consisting of personal information regarding the patient, information on findings shown by a medical examination performed on the patient, and medical history information regarding the patient.

### (Supplementary Note B3)

The symptom understanding assist method described in supplementary note B1 or B2, in which in the acquiring explanatory text, the at least one processor converts, into text data, a speech picked up in the medical interview performed on the patient.

### (Supplementary Note B4)

The symptom understanding assist method described in any one of supplementary notes B1 to B3, in which in the inferring, the at least one processor infers the symptom candidate, based on an output obtained by inputting the summary text and the medical information to a trained model generated by machine learning.

### (Supplementary Note B5)

The symptom understanding assist method described in any one of supplementary notes B1 to B4, in which the medical information includes a diagnosis target image of the patient.

### (Supplementary Note B6)

The symptom understanding assist method described in any one of supplementary notes B1 to B5, in which the symptom candidate is information to be used in decision making on diagnosis of the patient.

### Additional Remark C

The whole or part of the example embodiments disclosed above can be described as, but not limited to, the following supplementary notes.

### (Supplementary Note C1)

A symptom understanding assist program for causing a computer to function as a symptom understanding assist apparatus, the symptom understanding assist program causing the computer to function as:
an explanation acquiring means for acquiring explanatory text of an explanation made by a patient about a symptom in the patient;
a summary generating means for generating summary text of the explanatory text with use of a large language model with the explanatory text used as an input;
a medical information acquiring means for acquiring medical information regarding the patient;
a symptom inferring means for inferring, based on the summary text and the medical information, a symptom candidate for the symptom in the patient; and
an outputting means for outputting the summary text and the symptom candidate inferred.

### (Supplementary Note C2)

The symptom understanding assist program described in supplementary note C1, in which the medical information includes at least one selected from the group consisting of personal information regarding the patient, information on findings shown by a medical examination performed on the patient, and medical history information regarding the patient.

### (Supplementary Note C3)

The symptom understanding assist program described in supplementary note C1 or C2, in which the explanation acquiring means is configured to convert, into text data, a speech picked up in the medical interview performed on the patient.

### (Supplementary Note C4)

The symptom understanding assist program described in any one of supplementary notes C1 to C3, in which the symptom inferring means is configured to infer the symptom candidate, based on an output obtained by inputting the summary text and the medical information to a trained model generated by machine learning.

### (Supplementary Note C5)

The symptom understanding assist program described in any one of supplementary notes C1 to C4, in which the medical information includes a diagnosis target image of the patient.

### (Supplementary Note C6)

The symptom understanding assist program described in any one of supplementary notes C1 to C5, in which the symptom candidate is information to be used in decision making on diagnosis of the patient.

### Additional Remark D

The whole or part of the example embodiments disclosed above can be described as, but not limited to, the following supplementary notes.

### (Supplementary Note D1)

A symptom understanding assist apparatus, including
at least one processor, the at least one processor carrying out:
an explanation acquiring process of acquiring explanatory text of an explanation made by a patient about a symptom in the patient;
a summary generating process of generating summary text of the explanatory text with use of a large language model with the explanatory text used as an input;
a medical information acquiring process of acquiring medical information regarding the patient;
a symptom inferring process of inferring, based on the summary text and the medical information, a symptom candidate for the symptom in the patient; and
an outputting process of outputting the summary text and the symptom candidate inferred.

The symptom understanding assist apparatus may further include a memory. The memory may have stored therein a program for causing the at least one processor to carry out each of the processes.

### (Supplementary Note D2)

The symptom understanding assist apparatus described in supplementary note D1, in which the medical information includes at least one selected from the group consisting of personal information regarding the patient, information on findings shown by a medical examination performed on the patient, and medical history information regarding the patient.

### (Supplementary Note D3)

The symptom understanding assist apparatus described in supplementary note D1 or D2, in which in the explanation acquiring process, the at least one processor converts, into text data, a speech picked up in the medical interview performed on the patient.

### (Supplementary Note D4)

The symptom understanding assist apparatus described in any one of supplementary notes D1 to D3, in which in the symptom inferring process, the at least one processor infers the symptom candidate, based on an output obtained by inputting the summary text and the medical information to a trained model generated by machine learning.

### (Supplementary Note D5)

The symptom understanding assist apparatus described in any one of supplementary notes D1 to D4, in which the medical information includes a diagnosis target image of the patient.

### (Supplementary Note D6)

The symptom understanding assist apparatus described in any one of supplementary notes D1 to D5, in which the symptom candidate is information to be used in decision making on diagnosis of the patient.

### Additional Remark E

The whole or part of the example embodiments disclosed above can be described as, but not limited to, the following supplementary notes.

### (Supplementary Note E1)

A non-transitory recording medium having recorded thereon a symptom understanding assist program for causing a computer to function as a symptom understanding assist apparatus, the symptom understanding assist program causing the computer to carry out:
an explanation acquiring process of acquiring explanatory text of an explanation made by a patient about a symptom in the patient;
a summary generating process of generating summary text of the explanatory text with use of a large language model with the explanatory text used as an input;
a medical information acquiring process of acquiring medical information regarding the patient;
a symptom inferring process of inferring, based on the summary text and the medical information, a symptom candidate for the symptom in the patient; and
an outputting process of outputting the summary text and the symptom candidate inferred.

### REFERENCE SIGNS LIST

1: Symptom understanding assist apparatus
1A: Information processing apparatus
10A: Control section
11, 11A: Explanation acquiring section
12, 12A: Summary generating section
13, 13A: Medical information acquiring section
14, 14A: Symptom inferring section
15, 50A: Outputting section
15A: Output control section

1. A symptom understanding assist apparatus, comprising
at least one processor, the at least one processor carrying out:
an explanation acquiring process of acquiring explanatory text of an explanation made by a patient about a symptom in the patient;
a summary generating process of generating summary text of the explanatory text with use of a large language model with the explanatory text used as an input;
a medical information acquiring process of acquiring medical information regarding the patient;
a symptom inferring process of inferring, based on the summary text and the medical information, a symptom candidate for the symptom in the patient; and
an outputting process of outputting the summary text and the symptom candidate inferred.

2. The symptom understanding assist apparatus according to claim 1, wherein
the medical information includes at least one selected from the group consisting of personal information regarding the patient, information on findings shown by a medical examination performed on the patient, and medical history information regarding the patient.

3. The symptom understanding assist apparatus according to claim 1, wherein
in the explanation acquiring process, the at least one processor converts, into text data, a speech picked up in the medical examination performed on the patient.

4. The symptom understanding assist apparatus according to claim 1, wherein
in the symptom inferring process, the at least one processor infers the symptom candidate, based on an output obtained by inputting the summary text and the medical information to a trained model generated by machine learning.

5. The symptom understanding assist apparatus according to claim 1, wherein
the medical information includes a diagnosis target image of the patient.

6. The symptom understanding assist apparatus according to claim 1, wherein
the symptom candidate is information to be used in decision making on diagnosis of the patient.

7. A symptom understanding assist method, comprising:
at least one processor acquiring explanatory text of an explanation made by a patient about a symptom in the patient;
the at least one processor generating summary text of the explanatory text with use of a large language model with the explanatory text used as an input;
the at least one processor acquiring medical information regarding the patient;
the at least one processor inferring, based on the summary text and the medical information, a symptom candidate for the symptom in the patient; and
the at least one processor outputting the summary text and the symptom candidate inferred.

8. A computer-readable non-transitory recording medium having recorded thereon a symptom understanding assist program for causing a computer to function as a symptom understanding assist apparatus, the symptom understanding assist program causing the computer to carry out:
an explanation acquiring process of acquiring explanatory text of an explanation made by a patient about a symptom in the patient;
a summary generating process of generating summary text of the explanatory text with use of a large language model with the explanatory text used as an input;
acquiring process a medical information of acquiring medical information regarding the patient;
a symptom inferring process of inferring, based on the summary text and the medical information, a symptom candidate for the symptom in the patient; and
an outputting process of outputting the summary text and the symptom candidate inferred.
